SEQUENCE LISTING (48 sequences)

SEQ ID NO: 1 - DNA, 324 bp, Homo sapiens
  CDS (1)..(324)
gacatccagatgacccagtctccatcctccctgtctgcatctgtaggagaccgtgtcaccatcacttgccgggcaagtcagggtattaagatgaagttaaattggtaccagcagaaaccagggaaagcccctaagctcctgatctataatacttcccatttgcaaagtggggtcccatcacgtttcagtggcagtggatctgggacagatttcactctcaccatcagcagtctgcaacctgaagattttgctacgtactactgtcaacagcataagacgcgtcctgatacgttcggccaagggaccaaggtggaaatcaaacgg

SEQ ID NO: 2 - protein, 108 aa, Homo sapiens
DIQMTQSPSSLSASVGDRVTITCRASQGIKMKLNWYQQKPGKAPKLLIYNTSHLQSGVPSRFSGSGSGTDFTLTISSLQPEDFATYYCQQHKTRPDTFGQGTKVEIKR

SEQ ID NO: 3 - DNA, 33 bp, Homo sapiens
  CDS (1)..(33)
cgggcaagtcagggtattaagatgaagttaaat

SEQ ID NO: 4 - protein, 11 aa, Homo sapiens
RASQGIKMKLN

SEQ ID NO: 5 - DNA, 21 bp, Homo sapiens
  CDS (1)..(21)
taatacttcccatttgcaaag

SEQ ID NO: 6 - protein, 6 aa, Homo sapiens
YFPFAK

SEQ ID NO: 7 - DNA, 27 bp, Homo sapiens
  CDS (1)..(27)
caacagcataagacgcgtcctgatacg

SEQ ID NO: 8 - protein, 9 aa, Homo sapiens
QQHKTRPDT

SEQ ID NO: 9 - DNA, 324 bp, Homo sapiens
  CDS (1)..(324)
gacatccagatgacccagtctccatcctccctgtctgcatctgtaggagaccgtgtcaccatcacttgccgagcaagtcagaggatttttacgaggttagagtggtaccagcagaaaccagggaaagcccctatgctcctgatctataatgtttcccatttgcaaagtggggtcccatcacgtttcagtggcagtggatctgggacagatttcactctcaccatcagcagtctgcaacctgaagattttgctacgtactactgtcaacagactgagattattcctattacgttcggccaagggaccaaggtggaaatcaaacag

SEQ ID NO: 10 - protein, 108 aa, Homo sapiens
DIQMTQSPSSLSASVGDRVTITCRASQRIFTRLEWYQQKPGKAPMLLIYNVSHLQSGVPSRFSGSGSGTDFTLTISSLQPEDFATYYCQQTEIIPITFGQGTKVEIKQ

SEQ ID NO: 11 - DNA, 33 bp, Homo sapiens
  CDS (1)..(33)
cgagcaagtcagaggatttttacgaggttagag

SEQ ID NO: 12 - protein, 11 aa, Homo sapiens
RASQRIFTRLE

SEQ ID NO: 13 - DNA, 21 bp, Homo sapiens
  CDS (1)..(21)
taatgtttcccatttgcaaag

SEQ ID NO: 14 - protein, 6 aa, Homo sapiens
CFPFAK

SEQ ID NO: 15 - DNA, 27 bp, Homo sapiens
  CDS (1)..(27)
caacagactgagattattcctattacg

SEQ ID NO: 16 - protein, 9 aa, Homo sapiens
QQTEIIPIT

SEQ ID NO: 17 - DNA, 324 bp, Homo sapiens
  CDS (1)..(324)
gacatccagatgacccagtctccatcctccctgtctgcatctgtaggagaccgtgtcaccatcacttgccgggcaagtcaggcgattaagaataagttaggttggtaccagcagaaaccagggaaagcccctaagctcctgatctataaggcgtcctatttgcaaagtggggtcccatcacgtttcagtggcagtggatctgggacagatttcactctcaccatcagcagtctgcaacctgaagattttgctacgtactactgtcaacagatgaagtcgcgtcctaatacgttcggccaagggaccaaggtggaaatcaaacgg

SEQ ID NO: 18 - protein, 108 aa, Homo sapiens
DIQMTQSPSSLSASVGDRVTITCRASQAIKNKLGWYQQKPGKAPKLLIYKASYLQSGVPSRFSGSGSGTDFTLTISSLQPEDFATYYCQQMKSRPNTFGQGTKVEIKR

SEQ ID NO: 19 - DNA, 33 bp, Homo sapiens
  CDS (1)..(33)
cgggcaagtcaggcgattaagaataagttaggt

SEQ ID NO: 20 - protein, 11 aa, Homo sapiens
RASQAIKNKLG

SEQ ID NO: 21 - DNA, 21 bp, Homo sapiens
  CDS (1)..(21)
taaggcgtcctatttgcaaag

SEQ ID NO: 22 - protein, 6 aa, Homo sapiens
GVLFAK

SEQ ID NO: 23 - DNA, 27 bp, Homo sapiens
  CDS (1)..(27)
caacagatgaagtcgcgtcctaatacg

SEQ ID NO: 24 - protein, 9 aa, Homo sapiens
QQMKSRPNT

SEQ ID NO: 25 - DNA, 324 bp, Homo sapiens
  CDS (1)..(324)
gacatccagatgacccagtctccatcctccctgtctgcatctgtaggagaccgtgtcaccatcacttgccgggcaagtcagtggatttttactgagttagcgtggtaccagcagaaaccagggaaagcccctaagctcctgatctataatgcgtcctatttgcaaagtggggtcccatcacgtttcagtggcagtggatctgggacagatttcactctcaccatcagcagtctgcaacctgaagatttcgctacgtactactgtcaacagacttttacgcctcctattacgttcggccaagggaccaaggtggaaatcaaacgg

SEQ ID NO: 26 - protein, 108 aa, Homo sapiens
DIQMTQSPSSLSASVGDRVTITCRASQWIFTELAWYQQKPGKAPKLLIYNASYLQSGVPSRFSGSGSGTDFTLTISSLQPEDFATYYCQQTFTPPITFGQGTKVEIKR

SEQ ID NO: 27 - DNA, 33 bp, Homo sapiens
  CDS (1)..(33)
cgggcaagtcagtggatttttactgagttagcg

SEQ ID NO: 28 - protein, 11 aa, Homo sapiens
RASQWIFTELA

SEQ ID NO: 29 - DNA, 21 bp, Homo sapiens
  CDS (1)..(21)
taatgcgtcctatttgcaaag

SEQ ID NO: 30 - protein, 6 aa, Homo sapiens
CVLFAK

SEQ ID NO: 31 - DNA, 27 bp, Homo sapiens
  CDS (1)..(27)
caacagacttttacgcctcctattacg

SEQ ID NO: 32 - protein, 9 aa, Homo sapiens
QQTFTPPIT

SEQ ID NO: 33 - DNA, 324 bp, Homo sapiens
  CDS (1)..(324)
gacatccagatgacccagtctccatcctccctgtctgcatctgtaggagaccgtgtcaccatcacttgccgggcaagtcagtggatttttgatcggttacattggtaccagcagaaaccagggaaagcccctacgctcctgatctataatagttccacgttgcaaagtggggtcccatcacgtttcagtggcagtggatctgggacagattttactctcaccatcagcagtctgcaacctgaagattttgctacgtactactgtcaacagactaagtctcgtccttcgacgttcggccaagggaccaaggtggaaatcaaacgg

SEQ ID NO: 34 - protein, 108 aa, Homo sapiens
DIQMTQSPSSLSASVGDRVTITCRASQWIFDRLHWYQQKPGKAPTLLIYNSSTLQSGVPSRFSGSGSGTDFTLTISSLQPEDFATYYCQQTKSRPSTFGQGTKVEIKR

SEQ ID NO: 35 - DNA, 33 bp, Homo sapiens
  CDS (1)..(33)
cgggcaagtcagtggatttttgatcggttacat

SEQ ID NO: 36 - protein, 11 aa, Homo sapiens
RASQWIFDRLH

SEQ ID NO: 37 - DNA, 21 bp, Homo sapiens
  CDS (1)..(21)
taatagttccacgttgcaaag

SEQ ID NO: 38 - protein, 5 aa, Homo sapiens
FHVAK

SEQ ID NO: 39 - DNA, 27 bp, Homo sapiens
  CDS (1)..(27)
caacagactaagtctcgtccttcgacg

SEQ ID NO: 40 - protein, 9 aa, Homo sapiens
QQTKSRPST

SEQ ID NO: 41 - DNA, 324 bp, Homo sapiens
  CDS (1)..(324)
gacatccagatgacccagtctccatcctccctgtctgcatctgtaggagaccgcgtcaccatcacttgccgggcaagtcagaatattaagacgaagttaaattggtatcagcagaaaccagggaaagcccctaagctcctgatctatcgtgtttccactttgcaaagtggggtcccatcacgtttcagtggcagtggatctgggacagatttcactctcaccatcagcagtctgcaacctgaagattttgctacgtactactgtcaacagactaagaagaggccttttacgttcggccaagggaccaaggtggaaatcgaacgg

SEQ ID NO: 42 - protein, 108 aa, Homo sapiens
DIQMTQSPSSLSASVGDRVTITCRASQNIKTKLNWYQQKPGKAPKLLIYRVSTLQSGVPSRFSGSGSGTDFTLTISSLQPEDFATYYCQQTKKRPFTFGQGTKVEIER

SEQ ID NO: 43 - DNA, 33 bp, Homo sapiens
  CDS (1)..(33)
cgggcaagtcagaatattaagacgaagttaaat

SEQ ID NO: 44 - protein, 11 aa, Homo sapiens
RASQNIKTKLN

SEQ ID NO: 45 - DNA, 21 bp, Homo sapiens
  CDS (1)..(21)
tcgtgtttccactttgcaaag

SEQ ID NO: 46 - protein, 7 aa, Homo sapiens
SCFHFAK

SEQ ID NO: 47 - DNA, 27 bp, Homo sapiens
  CDS (1)..(27)
caacagactaagaagaggccttttacg

SEQ ID NO: 48 - protein, 9 aa, Homo sapiens
QQTKKRPFT

(19) United States
(12) Patent Application Publication
(10) Pub. No.: US 2010/0143371 A1
(43) Pub. Date: Jun. 10, 2010

(54) INTRABODIES

(76) Inventor: Zhenping Zhu, Oakland, NJ (US)

Correspondence Address: GlaxoSmithKline;GLOBAL PATENTS -US, UW2220, P. O. BOX 1539, KING OF PRUSSIA, PA, 19406-0939, US

(21) Appl. No.: 12/513,061

(22) PCT Filed: Oct. 31, 2007
(86) PCT No.: PCT/US07/83222

§ 371 Date: Jan. 29, 2010

Related U.S. Application Data
(60) Provisional application No. 60/855,901, filed on Oct. 31, 2006.

(51) Int. Cl. A61K 39/395; A61K 33/24; C12N 5/02; A61K 31/7088; C07K 16/40; A61P 35/04

(52) U.S. Cl. 424/158.1; 424/649; 435/375; 514/44 R; 530/387.1

(57) ABSTRACT

A single domain intrabody that binds to an intracellular protein or to an intracellular domain of an intracellular protein, such as Etk. Also provided is a method of inhibiting an intracellular enzyme, and treating a tumor in a patient by administering the intrabody or a nucleic acid expressing the inventive intrabody.

This application claims benefit of the priority of U.S. provisional application Ser. No. 60/855,901, filed on Oct. 31, 2006, the contents of which are incorporated by reference herein in their entirety.

### BACKGROUND OF THE INVENTION

Etk is a 70 kDa member of the Tec family of non-receptor protein tyrosine kinases, which also includes Btk, Itk, and Tec (Smith, C. I. et al, 2001, Bioessays 23:436-46.; Tomlinson, M. G. et al, 2004, Mol. Cell. Biol. 24:2455-66) and is expressed in a variety of hematopoietic, epithelial and endothelial cells and has been shown to be involved in several cellular processes including proliferation, differentiation and motility.

Etk, the endothelial and epithelial tyrosine kinase, is a member of the Tec family of non receptor tyrosine kinases. These kinases share a high degree of homology and typically contain an N-terminal pleckstrin homology (PH) domain, a Tec homology (TH) domain, an SH3 and SH2 domain and a C-terminal kinase catalytic domain (10). The expression of Tec family kinases has been primarily identified in hematopoietic cells (11-14). Etk, on the other hand, possesses a much broader expression profile than its counterparts. It was initially identified in bone marrow and subsequently found in epithelial cells, fibroblasts, and endothelial cells (15-18). Etk has been shown to be involved in various cellular processes including proliferation, differentiation, adhesion, motility, and survival (10, 18-22). Elevated expression of Etk has been reported in several aggressive metastatic carcinoma cell lines (19, 21, 23, 25, 26). The expression and activity of Etk is induced by growth factors, cytokines, G-protein-coupled receptors, the extracellular matrix, antigen receptors and possibly by hormones (10, 23, 24). For example, it has been reported that Src activates Etk in vivo through phosphorylation of Tyr-566. However, the role of Etk in cell growth and transformation remains to be determined.

Despite some early promising observations both in intro and in a number of disease models (6, 7, 36, 37), the main obstacle in the use of intrabodies remains those problems associated with their instability and tendency to aggregate when expressed inside the cell. These problems result from expression in the cytoplasm and concomitant lack of disulfide bond formation, leading to protein aggregation, insolubility, instability or incorrect folding (1, 38). The formation of disulfide bonds is usually critical for the structural integrity and function of the conventional antibody fragments, especially the single chain Fv (scFv), the most used antibody format in intrabody technology.

### SUMMARY OF THE INVENTION

Accordingly, the invention provides a single domain intrabody that binds to an intracellular (cytosolic) protein or intracellular domain of a protein. The intrabody is used to, e.g., specifically inhibit an enzymatic activity of the intracellular protein or domain. Intracellular proteins and domains that can be targets for the intrabody include kinases, a proteases, nucleases, telomerases, transferases, reductases, hydrolyases, and isomerases. In an embodiment of the invention, the intrabody is specific for a kinase domain. In another embodiment of the invention, the intrabody is specific for a kinase domain of a receptor tyrosine kinase. In another embodiment of the invention, the intrabody is specific for the kinase domain of Etk.

The invention further provides a method of selectively inhibiting an activity of an enzyme in a cell which comprises providing a single domain intrabody into the cell that binds to the enzyme and inhibits the enzymatic activity. The single domain intrabody can be provided into the cell by expressing a gene that encodes the single domain intrabody in the cell. Alternatively, the single domain intrabody can be provided into the cell by linking it to a membrane transfer peptide and contacting the hybrid protein with the cell.

In particular, the present invention provides for a single domain intrabody that binds to an intracellular protein or to an intracellular domain of an intracellular protein. The invention provides for a single domain intrabody that binds to an intracellular enzyme, such as, for example a single-domain intrabody that binds to a kinase domain of Etk.

In one embodiment, the invention provides a single domain intrabody that is a human kappa chain variable domain antibody comprising one, two, or three complementarity determining regions selected from the group consisting of SEQ ID NO:4, SEQ ID NO:6, and SEQ ID NO:8 at CDR1, CDR2, and CDR3 respectively.

In another embodiment, the invention provides a single domain intrabody that is a human kappa chain variable domain antibody comprising one, two, or three complementarity determining regions selected from the group consisting of SEQ ID NO:12, SEQ ID NO:14, and SEQ ID NO:16 at CDR1, CDR2, and CDR3 respectively.

In yet another embodiment, the invention provides for a single domain intrabody that is a human kappa chain variable domain antibody comprising one, two, or three complementarity determining regions selected from the group consisting of SEQ ID NO:20, SEQ ID NO:22, and SEQ ID NO:24 at CDR1, CDR2, and CDR3 respectively.

In yet another embodiment, the invention provides for a single domain intrabody that is a human kappa chain variable domain antibody comprising one, two, or three complementarity determining regions selected from the group consisting of SEQ ID NO:28, SEQ ID NO:30, and SEQ ID NO:32 at CDR1, CDR2, and CDR3 respectively.

In yet another embodiment, the invention provides for a single domain intrabody that is a human kappa chain variable domain antibody comprising one, two, or three complementarity determining regions selected from the group consisting of SEQ ID NO:36, SEQ ID NO:38, and SEQ ID NO:40 at CDR1, CDR2, and CDR3 respectively.

In yet another embodiment, the invention provides for a single domain intrabody that is a human kappa chain variable domain antibody comprising one, two, or three complementarity determining regions selected from the group consisting of SEQ ID NO:44, SEQ ID NO:46, and SEQ ID NO:48 at CDR1, CDR2, and CDR3 respectively.

In yet another embodiment, the invention provides for a single domain intrabody that is a human kappa chain variable domain antibody comprising a complementarity determining region selected from the group consisting SEQ ID NO:2, SEQ ID NO:10, SEQ ID NO:18, SEQ ID NO:26, SEQ ID NO:34, and SEQ ID NO:42.

Also provided is a method of selectively inhibiting intracellular enzyme activity in a cell by a method including administering or expressing the inventive intracellular protein in the cytoplasm of the cell. Preferably, the intracellular enzyme to be inhibited is a member of the Tec family of non receptor tyrosine kinases. Most preferably, the intracellular enzyme to be inhibited is Etk. The method preferably includes a step of introducing a nucleic acid molecule encoding the intracellular intrabody into a cell to be treated. The introduced nucleic acid molecule is one that is expressed in the cell, having a suitable art-known promoter for expressing the intrabody.

Also provided is a method of treating or inhibiting a tumor in a patient in need thereof. The inventive intrabody, or a nucleic acid expressing that intrabody, is administered to a patient in need of such treatment via a route selected from oral, intravenous, intraperitoneal, subcutaneous, and/or intramuscular administration. The method of treating or inhibiting a tumor optionally further comprises co-administering to the patient at least one additional anticancer therapeutic modality. The additional anticancer therapeutic modality is optionally an anti-neoplastic agent or radiation. The anti-neoplastic agent can be, e.g., an alkylating agent or an anti-metabolite. The alkylating agent is selected from, simply by way of example, cisplatin, cyclophosphamide, melphalan, and/or dacarbazine. The anti-metabolite is selected from, for example, doxorubicin, daunorubicin, paclitaxel and/or gemcitabine.

The invention further provides a method of inhibiting cell transformation by specifically inhibiting an enzyme in the cell with a single domain intrabody.

Also provided is a method of treating or ameliorating a disease by specifically inhibiting, e.g., selectively inhibiting, an enzyme in the cell with a single domain intrabody. In one embodiment of the invention, the disease is a neoplastic disease.

### BRIEF DESCRIPTION OF THE FIGURES

FIG. 1 shows Etk binding characteristics of single domain antibodies. Etk. Thirty-four individual single domain antibodies were expressed in E. coli BL21 cells. (A) Crude extracts containing the antibody were subjected to qualitative, ELISA-based, binding assay with immobilized recombinant Etk. (B) Crude extracts containing the antibody were subjected to qualitative, ELISA-based, in vitro Etk kinase inhibition assay. Results are mean±SD of 2 independent experiments.

FIG. 2 shows expression and characterization of the soluble single domain antibodies. Soluble single domain antibodies were expressed in E. coli BL21 cells and purified using protein L affinity chromatography. (A) Purified single domain antibody preparations were resolved by SDS-PAGE and gels were stained with coomassie-blue. (B) Indicated concentrations of purified single domain antibodies were subjected to quantitative, ELISA-based, binding assay with immobilized recombinant Etk. (C) Indicated concentrations of purified domain antibodies were subjected to quantitative, ELISA-based, in vitro Etk kinase inhibition assay. Results are mean±SD of 2 independent experiments done in duplicates.

FIG. 3 shows binding of intrabodies expressed in transfected NSR cells to endogenous Etk. K5, K7, K9, K11, K12 and K15 correspond to NSR cells transfected with pcDNA3.1-L5, L7, L9, L11, L12 and L15, respectively. (A) Total cell extracts of NSR and intrabody-transfected NSR cells were resolved on SDS-PAGE and immunoblotted with an anti-Etk or an anti-c-Myc antibody. (B) Intrabodies were immunoprecipitated from cell extracts using immobilized protein L. Immunocomplexes were resolved by SDS-PAGE and immunoblotted with an anti-Etk or an anti-c-Myc antibody. (C) Etk was immunoprecipitated from cell extracts using an immobilized anti Etk antibody. Immunocomplexes were resolved by SDS-PAGE and immunoblotted with an anti-Etk or an anti-c-Myc antibody. Results are representative of 3 independent experiments.

FIG. 4 shows in vitro kinase activity of Etk from NSR cells and intrabody-bound ETK from intrabody-transfected NSR cells. ETK and intrabody-bound Etk were immunoprecipitated using an immobilized Etk antibody. (A) Immunocomplexes were subject to in vitro autophosphorylation. [γ-³³P]-labeled-Etk was resolved by SDS-PAGE. Autoradiographs were quantitated by densitometry. Results are mean±SD of 2 independent experiments. (B) Immunocomplexes were used in an ELISA-based in vitro kinase assay as described in the text and substrate phosphorylation was determined. Results are mean±SD of 2 independent experiments done in duplicates.

FIG. 5 shows colony formation on soft-agar by NSR and intrabody-transfected NSR cells. Cells were plated (10⁵/plate) in soft-agar medium and grown for 14 days. Colonies were stained with MMT and counted using the AlphaEaseFC program. Results shown are the mean±SD of 3 independent experiments done in triplicates.

### DETAILED DESCRIPTION OF THE INVENTION

The invention provides intrabodies that consist of a single immunoglobulin variable domain. The intrabodies avoid the instability and tendency towards aggregation associated with larger immunoglobulin based proteins (e.g., scFvs) when subject to the intracellular environment. Further, the intrabodies are capable of specifically inhibiting a single enzyme or enzymatic activity in a cell.

Specific inhibition is particularly advantageous where is it desired to minimize undesired cross-reactivity with other cellular components that are similar to the target enzyme. For example, in research or clinical applications, it is usually desirable to inhibit a single tyrosine kinase or a single signal transduction pathway in a cell. Unfortunately, it is difficult to discover or design a small molecule inhibitor of a particular enzyme that is sufficiently specific so as not to be otherwise toxic when administered at a useful concentration. In contrast, intrabodies of the invention effectively inhibit only their target. Like any other antibody, intrabodies of the invention can be selected for affinity and specificity. Further, they can be selected under conditions that replicate the intracellular environment in which they will be employed.

The domain intrabodies of the invention bind to intracellular enzymes and reduce or inhibit enzymatic activity. Examples of intracellular enzymes and enzymatic domains of membrane-bound proteins whose activities can be modulated according to the invention include but are not limited to kinases, proteases, nucleases, telomerases, transferases, reductases hydrolyases, isomerases. For example, in an embodiment of the invention, an intrabody specific for the kinase domain of Etk binds to Etk and reduces or inhibits autophosphorylation or phosphorylation of a substrate. The substrate can be a natural substrate (i.e., a substrate that is a normal intracellular target) or any other substrate that is indicative of the normal physiological activity of the enzyme.

According to the invention, specific reduction or inhibition of Etk kinase activity results in reduced transformation of vSrc-expressing cells. Accordingly, Etk plays a role in cell proliferation and survival. Reduced Etk kinase activity limits Src-induced cellular transformation.

In an embodiment of the invention, a domain intrabody binds to the intracellular domain of a receptor tyrosine kinase and reduces or inhibits signal transduction activity. The reduction or inhibition of signal transduction activity can be determined by assaying autophosphorylation or substrate phosphorylation. Examples of receptor tyrosine kinases include, but are not limited to, epidermal growth factor (EGFR), insulin-like growth factor receptor (IGF-IR), platelet derived growth factor receptor-alpha and -beta (PDGFR-α and PDGFR-β), and vascular endothelial growth factor receptors (including VEGFR1, VEGFR2, and VEGFR3). For example, in an embodiment of the invention, an anti-IGF-IR domain intrabody reduces or inhibits autophosphorylation of the beta subunit of IGF-IR and/or phosphorylation of one or more IFG-IR substrates, such as MAPK, Akt, and IRS-1.

Inhibition of enzymatic activity can be determined in vivo, ex vivo, or in vitro using, for example, tissues, cultured cell, or purified cellular components by methods that are well known in the art. When the intracellular enzyme is a kinase, phosphorylation can be detected, for example, using an antibody specific for phosphotyrosine in an ELISA assay or on a western blot. Some assays for tyrosine kinase activity are described in Panek et al., J. Pharmacol. Exp. Thera. 283: 1433-44 (1997) and Batley et al., Life Sci. 62:143-50 (1998). In certain embodiments, intrabodies of the invention cause a decrease in autophosphorylation or substrate phosphorylation that is at least about 50%, or at least about 70%, or at least about 80%. The inhibition can be greater; but the physiological effects can be significant where there is only partial inhibition. Accordingly, in an embodiment of the invention, enzymatic inhibition can be less than about 95%, or less than about 90%, or less than about 85%. In one embodiment inhibition of receptor autophosphorylation is from about 50% to about 80%. In an embodiment of the invention, the domain antibody concentration that results in 50% inhibition of enzymatic activity measured in vitro is less than about 1 μM, or less than about 100 nM, or less than about 50 nM, or less than about 25 nM. In an embodiment of the invention, the IC₅₀ is in a range of 5 nM to 50 nM or in a range of 10 nM to 40 nM.

Inhibition can also be determined by observation of physiologic effects. For example, inhibition of a tyrosine kinase can result in inhibition, diminution, inactivation and/or disruption of growth (proliferation and differentiation), transformation (colony formation), angiogenesis (blood vessel recruitment, invasion, and metastasis), and cell motility and metastasis (cell adhesion and invasiveness).

In addition, methods for detection of protein expression can be utilized to determine enzymatic inhibition wherein expression of the proteins being measured is influenced by the enzymatic activity of intrabody target. These methods include immunohistochemistry (IHC) for detection of protein expression, fluorescence in situ hybridization (FISH) for detection of gene amplification, competitive radioligand binding assays, solid matrix blotting techniques, such as Northern and Southern blots, reverse transcriptase polymerase chain reaction (RT-PCR) and ELISA. See, e.g., Grandis et al., Cancer, 78:1284-92 (1996); Shimizu et al., Japan J. Cancer Res., 85:567-71 (1994); Sauter et al., Am. J. Path., 148:1047-53 (1996); Collins, Glia 15:289-96 (1995); Radinsky et al., Clin. Cancer Res. 1:19-31 (1995); Petrides et al., Cancer Res. 50:3934-39 (1990); Hoffmann et al., Anticancer. Res. 17:4419-26 (1997); Wikstrand et al., Cancer Res. 55:3140-48 (1995).

Ex vivo assays can also be utilized to determine enzymatic inhibition by the intrabody. Such assays can involve the modulation done or more phenotypes mediated by the enzyme. In some cases, the target enzyme will be sufficient active that inhibition will lead to an apparent change in a cellular characteristic, that can be observed or measured. For example, in a Src-transformed cell, inhibition of Etk can be observed as inhibition of growth in soft agar. In other cases, it will be necessary to overexpress or otherwise activate the target enzyme in a test cell so as to produce an observable or measurable characteristic, the inhibition of which can be detected. For example, receptor tyrosine kinase inhibition can be observed by mitogenic assays using cell lines stimulated with receptor ligand in the presence and absence of inhibitor. The MCF7 breast cancer line (American Type Culture Collection (ATCC), Rockville, Md.) is such a cell line that expresses IGF-IR and is stimulated by IGF-I or IGF-II. Inhibition can also be observed using tumor models, for example, human tumor cells injected into a mouse.

According to the invention, an intrabody is created that specifically inhibits an intracellular enzyme or enzymatic activity. The intrabody can be selected to bind to an intracellular enzyme of any organism. Notably, because of the degree of evolutionary conservation of enzymes, protein-protein interactions, and signal transduction pathways, even though the intrabody will preferably be specific for an enzyme in a cell, it can also be expected that the intrabody will inhibit a homologous enzyme in a related species. For example, as set forth below, intrabodies are created that bind to and inhibit the kinase domain of human Etk/Bmx. In the region of the kinase domain, the human Etk/Bmx (GenBank Accession No. AAC08966) and mouse Etk/Bmx (GenBank Accession No. NP_{—}033889) amino acid sequences are about 95% identical (244 out of 258 amino acids). Accordingly, many human Etk kinase domain specific intrabodies can be expected to bind to and inhibit the mouse Etk kinase domain.

Domain intrabodies specific for any particular enzyme or catalytic region thereof can be readily identified by screening a single domain antibody library. Antibody engineering has enabled the production of single domain antibody libraries, and such libraries have been constructed from a number of variable domain scaffolds, including human V_{H} or Y_{L} (Jespers, L. et al., 2004, J. Mol. Biol. 337:893-903), camelid V_{H} (Tanha, J. et al., 2001, J. Biol. Chem. 276:24774-80), and shark V-NAR (Nuttall, S. D., et al., 2004, Proteins 55:187-97). Libraries from other species exist as well. However, to avoid adverse immune responses when domain intrabodies are administered to a subject, it is generally preferable that source of domain intrabody correspond to the subject to which the intrabody will be administered. As described below for a domain antibody library selected for binding to Etk, over 90% of recovered clones after three rounds of selection are antigen specific.

In an embodiment of the invention, domain intrabodies are obtained by selecting a single variable domain from a variable region of an antibody having two variable domains (i.e., a heterodimer of a heavy chain variable domain and a light chain variable domain). Methods for obtaining heavy chain-light chain heterodimers include, for example, the immunological method described by Kohler and Milstein, Nature 256:495-497 (1975) and Campbell, Monoclonal Antibody Technology, The Production and Characterization of Rodent and Human Hybridomas, Burdon et al., Eds., Laboratory Techniques in Biochemistry and Molecular Biology, Volume 13, Elsevier Science Publishers, Amsterdam (1985); as well as by the recombinant. DNA methods such as described by Huse et al, Science 246, 1275-81. (1989). The antibodies can also be obtained from phage display or yeast surface display libraries bearing combinations of V_{H} and V_{L} domains in the form of scFv or Fab. The V_{H} and V_{L} domains can be encoded by nucleotides that are synthetic, partially synthetic, or naturally derived. Single variable domain antibodies can also be found in Fab and scFv phage display libraries (Cai, X. et al., 1996, Proc. Natl. Acad. Sci. USA. 93:6280-5). In certain embodiments, phage display libraries bearing human antibody fragments are preferred. Other sources of human antibodies are transgenic mice engineered to express human immunoglobulin genes.

The invention provides intrabodies having binding characteristics that have been improved by direct mutation or methods of affinity maturation. The same methods used for modifying or increasing affinity and specificity of antibody binding sites consisting of two variable domains can be applied to intrabodies (see, e.g., Yang et al., J. Mol. Biol. 254:392-403 (1995)). For example, libraries binding domains into which diversity has been introduced can be easily screened for desired binding characteristics using phage display. Alternatively, yeast surface display can be employed. Thus intrabodies can be modified or improved by mutating CDR and/or FW residues and screening for desired characteristics. One way to introduce diversity is to randomize individual amino acid residues or combinations of residues so that in a population of otherwise identical antigen binding sites, subsets of from two to twenty amino acids are found at particular positions. Alternatively, mutations can be induced over a range of residues by error prone PCR methods (see, e.g., Hawkins et al., J. Mol. Biol. 226: 889-96 (1992)). In another example, a phage display vector containing a heavy or light chain variable region gene can be propagated in a mutator strain of E. coli (see, e.g., Low et al., J. Mol. Biol. 250: 359-68 (1996)). These methods of mutagenesis are illustrative of the many methods known to one of skill in the art.

It can be desirable to remove the disulfide bond that might be present in the single-domain intrabody to make the stability and affinity of the intrabody independent of the oxidation state of its environment. In one embodiment, one cysteine is substituted with valine and the other cysteine is substituted with alanine.

Conservative amino acid substitutions based on size, charge, or hydrophobicity can also be made that effect non-binding characteristics such as solubility or transport across membranes. Conservative changes are made by substituting one or two amino acids with amino acids with generally similar properties (e.g., acidic, basic, aromatic, size, positively or negatively charged, polarity, non-polarity) such that the substitutions do not substantially alter characteristics (e.g., charge, isoelectric point, affinity, avidity, conformation, solubility) or activity that are desired to be maintained. Typical substitutions that may be performed for such conservative amino acid substitution may be among the groups of amino acids as follows:

glycine (G), alanine (A), valine (V), leucine (L) and isoleucine (I);

aspartic acid (D) and glutamic acid (E);

alanine (A), serine (S) and threonine (T);

histidine (H), lysine (K) and arginine (R):

asparagine (N) and glutamine (Q);

phenylalanine (F), tyrosine (Y) and tryptophan (W)

Conservative amino acid substitutions will generally be acceptable in regions flanking the hypervariable regions primarily responsible for the selective and/or specific binding characteristics of the intrabody.

An example of a domain intrabody of the present invention that binds to the kinase domain of Etk is a human kappa variable domain antibody having one, two, or three complementarity determining regions (CDRs) selected from the group consisting of SEQ ID NO:4, SEQ ID NO:6, and SEQ ID NO:8 at CDR1, CDR2, and CDR3 respectively. Another example has one, two, or three CDRs selected from the group consisting of SEQ ID NO:12, SEQ ID NO:14, and SEQ ID NO:16 at CDR1, CDR2, and CDR3 respectively. Table 1 provides the amino acid sequences of the aforementioned CDRs and the CDRs of four additional examples.

TABLE 1
Kappa variable domain CDRs

| Source | | | |
|---|---|---|---|
| L5 | CDR1 | RASQGIKMKLN | SEQ ID NO: 4 |
| | CDR2 | YFPFAK | SEQ ID NO: 6 |
| | CDR3 | QQHKTRPDT | SEQ ID NO: 8 |
| L7 | CDR1 | RASQRIFTRLQ | SEQ ID NO: 12 |
| | CDR2 | CFPFAK | SEQ ID NO: 14 |
| | CDR3 | QQTEIIPIT | SEQ ID NO: 16 |
| L9 | CDR1 | RASQAIKDKLG | SEQ ID NO: 20 |
| | CDR2 | GVLFAK | SEQ ID NO: 22 |
| | CDR3 | QQMKSRPDT | SEQ ID NO: 24 |
| L11 | CDR1 | RASQWIFTELA | SEQ ID NO: 28 |
| | CDR2 | CVLFAK | SEQ ID NO: 30 |
| | CDR3 | QQTFTPPIT | SEQ ID NO: 32 |
| L12 | CDR1 | RASQWIFDRLH | SEQ ID NO: 36 |
| | CDR2 | FHVAK | SEQ ID NO: 38 |
| | CDR3 | QQTKSRPST | SEQ ID NO: 40 |
| L15 | CDR1 | RASQNIKTKLN | SEQ ID NO: 44 |
| | CDR2 | SCFHFAK | SEQ ID NO: 46 |
| | CDR3 | QQTKKRPFT | SEQ ID NO: 48 |

In another embodiment, a domain intrabody of the present invention that binds to the Etk kinase domain comprises the kappa variable domain of SEQ ID NO:2, or SEQ ID NO:10, or SEQ ID NO:18, or SEQ ID NO:25, or SEQ ID NO:34, or SEQ ID NO:42.

In another embodiment, domain intrabodies of the invention compete for binding to Etk with any one or more of L5, L7, L9, L11, L12, and L15.

The present invention also provides isolated polynucleotides encoding the domain intrabodies described. Accordingly, the invention includes nucleic acids having a sequence encoding one, two, or all three CDRs as, set forth in Table 2.

TABLE 2
Kappa variable domain CDRs

Source

| | | | |
|---|---|---|---|
| L5 | CDR1 | cgggcaagtc agggtattaa gatgaagtta | SEQ ID |
| | | aat | NO: 3 |
| | CDR2 | taatacttcc catttgcaaa g | SEQ ID |
| | | | NO: 5 |
| | CDR3 | caacagcata agacgcgtcc tgatacg | SEQ ID |
| | | | NO: 7 |
| L7 | CDR1 | cgagcaagtc agaggatttt tacgaggtta | SEQ ID |
| | | gag | NO: 11 |
| | CDR2 | taatgtttcc catttgcaaa g | SEQ ID |
| | | | NO: 13 |
| | CDR3 | caacagactg agattattcc tattacg | SEQ ID |
| | | | NO: 15 |
| L9 | CDR1 | cgggcaagtc aggcgattaa gaataagtta | SEQ ID |
| | | ggt | NO: 19 |
| | CDR2 | taaggcgtcc tatttgcaaa g | SEQ ID |
| | | | NO: 21 |
| | CDR3 | caacagatga agtcgcgtcc taatacg | SEQ ID |
| | | | NO: 23 |
| L11 | CDR1 | cgggcaagtc agtggatttt tactgagtta | SEQ ID |
| | | gcg | NO: 27 |
| | CDR2 | taatgcgtcc tatttgcaaa g | SEQ ID |
| | | | NO: 29 |
| | CDR3 | caacagactt ttacgcctcc tattacg | SEQ ID |
| | | | NO: 31 |
| L12 | CDR1 | cgggcaagtc agtggatttt tgatcggtta | SEQ ID |
| | | cat | NO: 35 |
| | CDR2 | taatagttcc acgttgcaaa g | SEQ ID |
| | | | NO: 37 |
| | CDR3 | caacagacta agtctcgtcc ttcgacg | SEQ ID |
| | | | NO: 39 |
| L15 | CDR1 | cgggcaagtc agaatattaa gacgaagtta | SEQ ID |
| | | aat | NO: 43 |
| | CDR2 | tcgtgtttcc actttgcaaa g | SEQ ID |
| | | | NO: 45 |
| | CDR3 | caacagacta agaagaggcc ttttacg | SEQ ID |
| | | | NO: 47 |

A domain intrabody of the invention can be used in methods designed to express the intrabody intracellularly so as to inhibit an intracellular enzyme. Such methods comprise delivering to a cell a domain intrabody which may be in any form used by one skilled in the art, for example, a protein, an RNA molecule which is translated, or a DNA vector which is transcribed and translated, wherein said intrabody binds to an inhibits an intracellular component of the cell.

In instances where a nucleic acid molecule encoding a domain intrabody is used, techniques known in the art may be used for cloning of the nucleic acid molecule into an expression vector. Methods commonly known in the art of recombinant DNA technology which can be used are described in Ausubel et al. (eds.), 1993, Current Protocols in Molecular Biology, John. Wiley & Sons, NY; and Kriegler, 1990, Gene Transfer and Expression, A Laboratory Manual, Stockton Press, N.Y.

The DNA encoding the domain intrabody of interest may be recombinantly engineered into a variety of host vector systems that also provide for replication of the DNA in large scale and contain the necessary elements for directing the transcription of the intrabody. The use of such a construct to transfect target cells in the patient will result in transcription of sufficient amounts of the intrabody to reduce or inhibit an enzymatic activity of the intrabody target. For example, a vector can be introduced in vivo such that it is taken up by a cell and directs the transcription of the intrabody molecule. Such a vector can remain episomal or become chromosomally integrated, as long as it can be expressed to produce the desired intrabody. Such vectors can be constructed by recombinant DNA technology methods standard in the art.

Vectors encoding the domain intrabody of interest can be plasmid, viral, or others known in the art, used for replication and expression in mammalian cells. Expression of the sequence encoding the domain intrabody can be regulated by any promoter/enhancer sequences known in the art to act in mammalian, preferably human cells. Such promoters/enhancers can be inducible or constitutive. Such promoters include but are not limited to the SV40 early promoter region (Benoist, C. and Chambon, P. 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the viral CMV promoter, the human β-chorionic gonadotropin-6 promoter (Hollenberg et al., 1994, Mol. Cell. Endocrinology 106:111-119), etc. In one embodiment, cell type specific promoter/enhancer sequences may be used to promote the synthesis of domain intrabody in particular cells or tissue types.

Vectors for use in the practice of the invention include any eukaryotic expression vectors, including but not limited to viral expression vectors such as those derived from the class of retroviruses, adenoviruses or adeno-associated viruses.

Various delivery systems are known and can be used to transfer the compositions, of the invention into cells, e.g., encapsulation in liposomes, microparticles, microcapsules, recombinant cells capable of expressing the composition, receptor-mediated endocytosis (see, e.g., Wu and Wu, 1987, J. Biol. Chem. 262:4429-4432), construction of a nucleic acid as part of a retroviral, adenoviral, adeno-associated viral or other vector, injection of DNA, electroporation, calcium phosphate mediated transfection, etc.

In one embodiment, nucleic acids comprising a sequence encoding a domain intrabody are administered to promote intrabody function, by way of gene delivery and expression into a host cell. In this embodiment of the invention, the nucleic acid mediates an effect by promoting intrabody production. Any of the methods for gene delivery into a host cell available in the art can be used according to the present invention. For general reviews of the methods of gene delivery see Strauss, M. and Barranger, J. A., 1997, Concepts in Gene Therapy, by Walter de Gruyter & Co., Berlin; Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; and Morgan and Anderson, 1993, Ann. Rev. Biochem. 62:191-217; 1993, TIBTECH 11(5):155-215.

In a specific embodiment, the nucleic acid encoding the inventive intrabody is directly administered in vivo, under conditions effective for production of an inventive intrabody. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, or by administering it in linkage to a ligand subject to receptor-mediated endocytosis (see e.g., Wu and Wu, 1987, J. Biol. Chem. 262:4429-4432).

In a specific embodiment, a viral vector that contains the intrabody can be used. For example, a retroviral vector can be utilized that has been modified to delete retroviral sequences that are not necessary for packaging of the viral genome and integration into host cell DNA (see Miller et al., 1993, Meth. Enzymol. 217:581-599). Alternatively, adenoviral or adeno-associated viral vectors can be used for gene delivery to cells or tissues. (See, Kozarsky and Wilson, 1993, Current Opinion in Genetics and Development 3:499-503 for a review of adenovirus-based gene delivery).

In a preferred embodiment of the invention, an adeno-associated viral vector may be used to deliver nucleic acid molecules that encode the intrabody. The vector is designed so that, depending on the level of expression desired, a promoter and/or enhancer element of choice may be inserted into the vector.

Another approach to gene delivery into a cell involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. The resulting recombinant cells can be delivered to a host by various methods known in the art. In a preferred embodiment, the cell used for gene delivery is autologous to the host cell.

Intrabodies may be fused or conjugated to a domain or sequence that has translocation activity. For example, the signal peptide of Kaposi fibroblast growth factor (Delli Bovi, P. et al., 1987, Cell 50:729-37) contains a hydrophobic sequence (AAVLLPVLLAAP) that functions as a cellular import signal (Shin, I. et al., 2005, Cancer Res. 65:2815-24) and can be fused at the N terminus of the intrabody. Thus, intrabodies can be produced outside a target cell and then added to a cell culture or administered to a subject.

Translocation activity has also been identified in amino acids 37-72 (Fawell et al., 1994, Proc. Natl. Acad. Sci. U.S.A. 91:664-8), amino acids 37-62 (Anderson et al., 1993, Biochem. Biophys. Rex. Commun. 194:876-84) and amino acids 49-58 (having the basic sequence RKKRRQRRR) of HIV-Tat. A longer peptide of HIV-Tat (amino acids 48-60; CGRKICRRQRRRPPQC) can be used for translocation, nuclear localization and trans-activation of cellular genes (Vives et al., 1997, J. Biol. Chem. 272:16010-7). Administration of a fusion protein containing an HIV-Tat translocation sequence and β-galactosidase resulted in delivery of active fusion protein to all tissues of a mouse (Schwarze et al., 1999, Science, 285:1569-72). A 16 amino acid basic peptide from the Drosophila antennapedia homeodomain protein (RQIIKIWFQNRRMKWKIC; Derossi, et al., 1994, J. Biol. Chem. 269:10444-50) can also be used to direct intrabodies to the cytoplasm of cells (Theodore, et al., 1995, J. Neurosci. 15:715867).

It is understood that the domain intrabodies of the invention, where used in a mammal for the purpose of prophylaxis or treatment, will be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers can further comprise minor amounts of auxiliary substances such as wetting or emulsifying agents, preservatives or buffers, which enhance the shelf life or effectiveness of the binding proteins. The compositions of the injection can, as is well known in the art, be formulated so as to provide quick, sustained or delayed release of the active ingredient after administration to the mammal.

In an embodiment of the invention where a domain intrabody is administered to or expressed in tissue of a subject with a neoplastic disease, one or more other anti-neoplastic agents can be coadministered. For examples of combination therapies, see, e.g., U.S. Pat. No. 6,217,866 (Schlessinger et al.) (Anti-EGFR antibodies in combination with anti-neoplastic agents); WO 99/60023 (Waksal et al.) (Anti-EGFR antibodies in combination with radiation). Any suitable anti-neoplastic agent can be used, such as a chemotherapeutic agent, radiation or combinations thereof. The anti-neoplastic agent can be an alkylating agent or an anti-metabolite. Examples of alkylating agents include, but are not limited to, cisplatin, cyclophosphamide, melphalan, and dacarbazine. Examples of anti-metabolites include, but not limited to, doxorubicin, daunorubicin, and paclitaxel, gemcitabine.

Useful anti-neoplastic agents also include mitotic inhibitors, such as taxanes docetaxel and paclitaxel. Topoisomerase inhibitors are another class of anti-neoplastic agents that can be used in combination with antibodies of the invention. These include inhibitors of topoisomerase I or topoisomerase II. Topoisomerase I inhibitors include irinotecan (CPT-11), aminocamptothecin, camptothecin, DX-8951f, topotecan. Topoisomerase II inhibitors include etoposide (VP-16), and teniposide (VM-26). Other substances are currently being evaluated with respect to topoisomerase inhibitory activity and effectiveness as anti-neoplastic agents. In a preferred embodiment, the topoisomerase inhibitor is irinotecan (CPT-11).

When the anti-neoplastic agent is radiation, the source of the radiation can be either external (external beam radiation therapy—EBRT) or internal (brachytherapy—BT) to the patient being treated. The dose of anti-neoplastic agent administered depends on numerous factors, including, for example, the type of agent, the type and severity tumor being treated and the route of administration of the agent. It should be emphasized, however, that the present invention is not limited to any particular dose.

Domain intrabodies of the invention can be coadministered with antibodies or other antagonists that neutralize receptors involved in tumor growth or angiogenesis. In an embodiment of the invention, an intrabody is expressed or administered in combination with a receptor antagonist that binds to EGFR. RTK antagonists also include antibodies or other agents, that bind to a ligand of the RTK and inhibits binding of the RTK to its ligand. Ligands for EGFR include, for example, EGF, TGF-α, amphiregulin, heparin-binding EGF (HB-EGF) and betacellulin. EGF and TGF-α are thought to be the main endogenous ligands that result in EGFR-mediated stimulation, although TGF-α has been shown to be more potent in promoting angiogenesis. EGFR antagonists also include substances that inhibit EGFR dimerization with other EGFR receptor subunits (i.e., EGFR homodimers) or heterodimerization with other growth factor receptors (e.g., HER2). EGFR antagonists further include biological molecules and small molecules, such as synthetic kinase inhibitors that act directly on the cytoplasmic domain of EGFR to inhibit EGFR-mediated signal transduction. Erbitux® (cetuximab) is an example of an EGFR antagonist that binds to EGFR and blocks ligand binding. One example of a small molecule EGFR antagonist is IRESSA™ (ZD1939), which is a quinozaline derivative that functions as an ATP-mimetic to inhibit EGFR. See U.S. Pat. No. 5,616,582 (Zeneca Limited); WO 96/33980 (Zeneca Limited) at p. 4; see also, Rowinsky et al., Abstract 5 presented at the 37th Annual Meeting of ASCO, San Francisco, Calif., 12-15 May 2001; Anido et al., Abstract 1712 presented at the 37th Annual Meeting of ASCO, San Francisco, Calif., 12-15 May 2001. Another example of a small molecule EGFR antagonist is TARCEVA™ (OSI-774), which is a 4-(substitutedphenylamino)quinozaline derivative [6,7-Bis(2-methoxy-ethoxy)-quinazolin-4-yl]-(3-ethynyl-phenyl)amine hydrochloride] EGFR inhibitor. See WO 96/30347 (Pfizer Inc.) at, for example, page 2, line 12 through page 4, line 34 and page 19, lines 14-17. See also Moyer et al., Cancer Res., 57: 4838-48 (1997); Pollack et al., J. Pharmacol., 291: 739-48 (1999). TARCEVA™ may function by inhibiting phosphorylation of EGFR and its downstream PI3/Akt and MAP (mitogen activated protein) kinase signal transduction pathways resulting in p27-mediated cell-cycle arrest. See Hidalgo et al., Abstract 281 presented at the 37th Annual Meeting of ASCO, San Francisco, Calif., 12-15 May 2001.

In another embodiment of the invention, an intrabody is expressed or administered in combination with a receptor antagonist that binds to IGF-IR. For example, IMC-A12 is a human antibody that binds to and neutralizes IGF-IR (WO2005016970; Ludwig). IGF-IR antagonists include but are not limited to antibodies that bind to IGF-IR or an IGF-IR ligand (e.g., IGF-1 and IFG-1). Small molecule antagonists of IGF-IR include, for example, the insulin-like growth factor-I receptor selective kinase inhibitors NVP-AEW541 (García-Echeverría, C. et al., 2004, Cancer Cell 5:231-9) and NVP-ADW742 (Mitsiades, C. et al., 2004, Cancer Cell 5:221-30), INSM-18 (Insmed Incorporated), which is reported to selectively inhibit IGF-IR and HER2, and the tyrosine kinase inhibitor tryphostins AG1024 and AG1034 (Párrizas, M. et al., 1997, Endocrinology 138:1427-33) which inhibit phosphorylation by blocking substrate binding and have a significantly lower IC₅₀ for inhibition of IFG-IR phosphorylation than for IR phosphorylation. The cyclolignan derivative picropodophyllin (PPP) is another IGF-IR antagonist that inhibits IGF-IR phosphorylation without interfering with IR activity (Girnita, A. et al., 2004, Cancer Res. 64:236-42). Other small molecule IGF-IR antagonists include the benzimidazol derivatives BMS-536924 (Wittman, M. et al., 2005, J. Med. Chem. 48:5639-43) and BMS-554417 (Haluska P. et al., 2006, Cancer Res. 66:362-71), which inhibit IGF-IR and IR almost equipotently. For compounds that inhibit receptors in addition to IGF-IR, it should be noted that IC₅₀ values measured in vitro in direct binding assays may not reflect IC₅₀ values measured ex vivo or in vivo (i.e., in intact cells or organisms). For example, where it is desired to avoid inhibition of IR, a compound that inhibits IR in vitro may not significantly affect the activity of the receptor when used in vivo at a concentration that effectively inhibits IGF-IR.

In an embodiment of the invention, a domain intrabody is expressed or administered in combination with a VEGFR antagonist. The VEGFR can be the VEGFR-1/Flt-1 receptor or the VEGFR-2/ICDR receptor. Particularly preferred are antigen-binding proteins that bind to the extracellular domain of VEGFR-1 or VEGFR-2 and block binding by their ligands (VEGFR-2 is stimulated most strongly by VEGF; VEGFR-1 is stimulated most strongly by P1GF, but also by VEGF) and/or neutralize ligand-induced induced activation. For example, IMC-1121 is a human antibody that binds to and neutralizes VEGFR-2 (WO 03/075840; Zhu). Another example is MAb 6.12 is a scFv that binds to soluble and cell surface-expressed VEGFR-1. ScFv 6.12 comprises the V_{L} and V_{H} domains of mouse monoclonal antibody MAb 6.12. A hybridoma cell line producing MAb 6.12 has been deposited as ATCC number PTA-3344 under the provisions of the Budapest Treaty on the International Recognition of the Deposit of Microorganisms for the Purposes of Patent Procedure and the regulations thereunder (Budapest Treaty). In another embodiment, an intrabody is used in combination with a receptor antagonist that binds to a VEGFR ligand and blocks activation of a VEGFR by the ligand. For example, Avastin® (bevacizumab) is an antibody that binds VEGF.

Other examples of growth factor RTKs involved in tumorigenesis are the receptors for platelet-derived growth factor (PDGFR), nerve growth factor (NGFR), and fibroblast growth factor (FGFR).

The intrabodies can also be used for patients who receive adjuvant hormonal therapy (e.g., for breast cancer) or androgen-deprivation therapy (e.g., for prostate cancer).

In a combination therapy, an inhibitory domain intrabody is administered before, during, or after commencing therapy with another agent, as well as any combination thereof, i.e., before and during, before and after, during and after, or before, during and after commencing the anti-neoplastic agent therapy. For example, the intrabody can be administered between 1 and 30 days, preferably 3 and 20 days, more preferably between 5 and 12 days before commencing radiation therapy. In a preferred embodiment of the invention, chemotherapy is administered concurrently with or, subsequent to antibody therapy.

In the present invention, any suitable method or route can be used to administer an intrabody of the invention, and optionally, to co-administer anti-neoplastic agents and/or antagonists of other receptors. The anti-neoplastic agent regimens utilized according to the invention, include any regimen believed to be optimally suitable for the treatment of the patient's neoplastic condition. Different malignancies can require use of specific anti-tumor antibodies and specific anti-neoplastic agents, which will be determined on a patient to patient basis. Routes of administration include, for example, oral, intravenous, intraperitoneal, subcutaneous, or intramuscular administration. The dose of antagonist administered depends on numerous factors, including, for example, the type of antagonists, the type and severity tumor being treated and the route of administration of the antagonists. It should be emphasized, however, that the present invention is not limited to any particular method or route of administration.

Throughout this application, various publications, reference texts, textbooks, technical manuals, patents, and patent applications have been referred to. The teachings and disclosures of these publications, patents, patent applications and other documents in their entireties are hereby incorporated by reference into this application to more fully describe the state of the art to which the present invention pertains.

It is to be understood and expected that variations in the principles of invention herein disclosed may be made by one skilled in the art and it is intended that such modifications are to be included within the scope of the present invention.

The following examples further illustrate the invention, but should not be construed to limit the scope of the invention in any way. Detailed descriptions of conventional methods, such as those employed in the construction of vectors and plasmids, and expression of antibodies and antibody fragments can be obtained from numerous publications, including Sambrook, J et al., (1989) Molecular Cloning: A Laboratory Manual, 2^{nd} ed., Cold Spring Harbor Laboratory Press; Coligan, J. et al. (1994) Current Protocols in Immunology, Wiley & Sons, Incorporated; Enna, S. J. et al. (1991) Current Protocols in Pharmacology, Wiley & Sons, Bonifacino, J. S. et al. (1999) Current Protocols in Cell Biology, Wiley & Sons. All references mentioned herein are incorporated by reference in their entireties.

### EXAMPLES

Library, Cells and Reagents.—A human domain antibody phage display library was obtained from Domantis (Cambridge, UK). NSR cells (mouse NIH3T3 cells over-expressing v-Src) were a gift from Dr. J. E. Darnell (Rockefeller University, New York, N.Y.). ATP, proteases inhibitor cocktail, 3-(4,5-dimethylthiazol-2-yl)-2,5-diphenyltetrazolium bromide (MTT), glutathione beads, tetracycline, trypsin and polyEY were purchased from Sigma Chemicals Co. (St. Louis, Mo.). Isopropyl-1-thio-β-D-galactopyranoside (IPTG), monoclonal anti-Myc-horseradish peroxidase (HRP) conjugate, low melting agar, Geneticin G418, NuPage polyacrylamide gel and transfer system, Lipofectamine 2000 and OptiMEM were from Invitrogen (Carlsbad, Calif.). Anti-M13-HRP antibodies and ³³P-γATP were from Amersham (Piscataway, N.J.). Monoclonal pY20 antibodies were obtained from Oncogene-EMD Biosciences (San Diego, Calif.). Polyclonal Etk antibodies were from Cell Signaling (Beverly, Mass.). Glutathione-S-transferase (GST) microbeads and μMacs column were from Milteny Biotec (Auburn, Calif.). AffinityPak immobilized protein L and protein A were purchased from Pierce (Rockford, Ill.). TMB peroxidase substrate was from KPL (Gaithersburg, Md.).

Generation of GST-Etk kinase domain. The gene fragment encoding Etk kinase domain (amino acid 417-697) was amplified and subcloned into pBac vector (Pharmingen, San Diego). Plasmid was used to infect SF21 cells using the baculovirus system, and a soluble fusion protein, GST-Etk kinase domain (GST-Etk), was expressed (Ellis, L. et al., J. Virol 62:1634-9, 1988). The kinase was purified using a Glutathione bead column (McNew, J. A. et al., Nature 407:153-9, 2000) and enzymatic activity of recombinant protein was confirmed by an in-vitro kinase assay (details below). GST peptide was generated using the same procedure.

Selection of anti-Etk Domain Antibodies from phage display library. A large domain phage display library, derived from a single human framework of light chain, containing 1.7×10¹⁰ clones was used for the selection. The library was generated by using side chain diversification incorporated at positions in the antigen binding regions, known to be highly diverse in the mature antibody repertoire, with complete randomized at 13 residues. In displaying the domain antibodies on filamentous phage surface, the PCR-amplified variable light chain domain gene was preceded by a signal sequence of GAS leader at the 5′ end. An 11 amino-acid long c-Myc tag was inserted between the C-terminal of the light chain variable region and gene III, for purification and detection purposes.

Library stock containing 10¹¹ phage units was resuspended in 1 ml PBS containing 3% fat-free milk, mixed with 5 μg of GST for 1 h at 37° C., to capture phage displaying anti-GST antibodies and to block other nonspecific binding, and followed by incubation with 100 μl of anti-GST magnetic beads for additional 30 min at RT. The mixture was loaded on a μMacs column and the flow-through was collected. Phage preparation, derived from flow through, was then mixed with 5 μg of GST-Etk for 1 h at RT, followed by incubation with 100 μl of anti-GST magnetic beads for additional 30 min at RT. The beads-GST-Etk-phage mix was loaded on a μMacs column, followed by 5 washes with 1 ml of PBS containing 0.1% Tween-20 (PBST), 5 washes with 1 ml of PBS, and elution of the bound phages by 500 μl of freshly prepared solution of 1 mg/ml trypsin. The eluted phage was incubated with 4 ml of mid-log phase E. coli TG1 cells for 30 min at 37° C. TG1 cells were spun down, resuspended and plated onto several 90 mm TYE plates containing 15 μg/ml tetracycline, and incubated overnight at 30° C. All the colonies grown on the plates were scraped into 2 ml of 2YT medium, mixed with glycerol to a final concentration of 15%, aliquoted and stored at −70° C. For further selection rounds, phage was amplified as previously described (Lu, D. et al, Int. J. Cancer 97:393-9, 2002) and 10¹¹ phage units were used for selection using the procedure described above, with decreasing amount of GST-Etk (2 μg and 1 μg for the 2^{nd} and the 3^{rd} rounds, respectively), along with more extensive washes (10 to 15 times) of the GST-Etk-phage-loaded μMacs columns.

A total of 3 rounds of selection were performed using recombinant GST-Etk, with alternating protein concentrations and number of washes after the antigen/antibody binding event. Following the 3^{rd} selection round, 540 clones were randomly picked and tested by phage ELISA for binding to both GST and GST-Etk.

Individual E. coli TG1 clones were picked and grown overnight at 37° C. in 2TY medium, supplemented with 15 μg/ml tetracycline, in a 96 well plate format. Bacteria culture was spun down and supernatant containing phage was mixed with fat-free milk to a final concentration of 3%, and incubated 1 h at RT. Phage was transferred to Maxi-sorp 96 well microtiter plates (Nunc, Roskilde, Denmark) coated with 100 μl/well of 1 μg/ml GST or GST-Etk, and incubated for 1 h at RT. The plates were washed 3 times with PEST and incubated with M13-HRP antibodies for additional 1 h at RT. The plate was washed 5 times, TMB peroxidase substrate was added and the absorbance at 450 nm was measured using a microplate reader (Molecular Devices, Sunnyvale, Calif.).

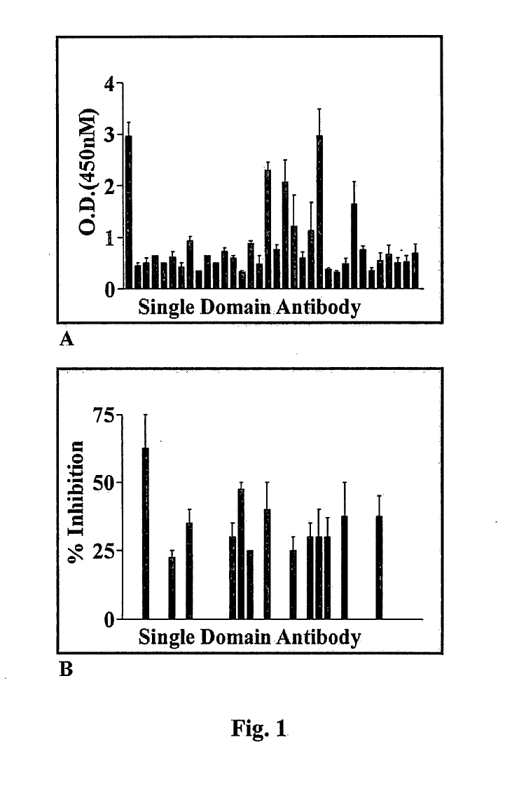

Over 90% ( 491/540) of the tested clones were positive and specific for Etk binding, suggesting a high efficiency of the selection process. DNA segments encoding the single domain antibodies, derived from the third selection, were then pooled, amplified, and subcloned into pDOM5 vector, for the expression of soluble proteins. Following transformation, individual E. coli BL21 clones were grown, in 96-well plates and induced for expression of domain antibodies with IPTG. Of 180 randomly picked colonies, 64 (35%), were positive for Etk binding as determined by a soluble antibody ELISA. Thirty-four binders (FIG. 1A) were further assayed for their capability in inhibiting Etk kinase, using polyEY as the substrate; 13 showed moderate to strong enzyme-blocking activity (FIG. 1B). Sequence analysis of the 20 best binders revealed 20 different patterns, indicating an excellent diversity of the isolated anti-Etk domain, antibodies.

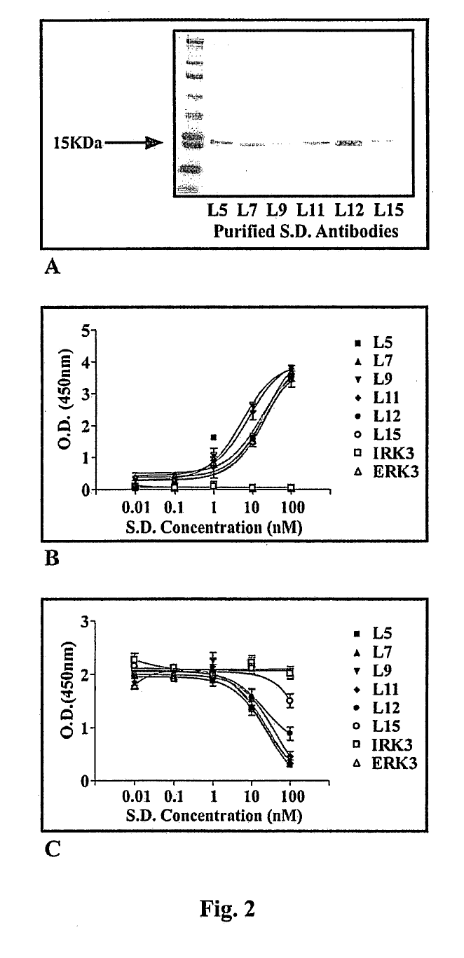

Expression and purification of Soluble Domain Antibody. To further study the binding and blocking characteristics of the domain antibodies toward Etk, 6 clones, L5, L7, L9, Lit, L12 and L15 were grown in a large scale culture and IPTG-induced to express soluble domain antibodies. DNA coding for domain antibodies were amplified and subcloned into the Sal I and Not I sites of an expression vector, pDOM5 (Domantis). E. coli BL21 cells transformed with individual expression vector were grown at 37° C. in 100 ml LB medium supplemented with 100 μg/ml ampicillin. When OD₆₀₀ reached ˜0.5, IPTG was added to a final concentration of 1 mM, and incubation was continued overnight at 30° C. Cells were spun down and the pellet was resuspended in 2 ml binding buffer composed of 100 mM Na₂HPO₄ and 150 mM NaCl pH 7.2, supplemented with 1:100 ratio of protease inhibitor cocktail. The cell suspension was sonicated, centrifuged, and the supernatant was collected and incubated with 100 μl protein L beads. The beads were washed with 20 ml binding buffer, followed by the elution of the single domain antibodies using 450 μl of 0.1M sodium citrate pH 3.1. The eluant was immediately mixed with 50 μl of neutralizing buffer containing 1M Tris-HCl, pH 8.0. The yield of purified antibodies ranged from 50-500 μg per 100 ml culture. SDS-PAGE analysis of each purified antibody preparation demonstrated a single protein band corresponding to the expected molecular size of 15 kDa (FIG. 2A).

Quantitative Etk binding assay. Various concentrations of purified domain antibodies were added into a Maxi-sorp 96 well microtiter plate coated with 100 μl/well of 1 μg/ml GST-Etk and incubated for 1 h at RT. The plate was washed 3 times with PBST and then incubated with 100 μl/well of anti-Myc-HRP conjugate (1:5000 dilution) for 1 h at RT. The plate was washed 5 times, TMB peroxidase substrate was added and the absorbance at 450 nm was measured.

As shown in FIG. 2B, all 6 domain antibodies bound Etk in a dose-dependent manner, with L9 as the best binder followed by L11, L5, L7, L12 and L15. The antibody concentration required for 50% of maximum binding to Etk was 5 nM and 8 nM for L9 and L11, respectively, and approximately 20 nM for L5, L7, L12 and L15. The control, non-relevant domain antibodies, IRK3 and ERK3, did not bind to Etk even at high doses (FIG. 2B). Consistent with the binding data, all 6 domain antibodies blocked Etk enzymatic activity dose-dependently, as indicated by the reduction in the ability of recombinant Etk to phosphorylate the substrate, polyEY, in the presence of the domain antibodies (FIG. 2C). The domain antibody concentration required to achieve 50% inhibition of Etk kinase activity was approximately 20 nM for L5, L7, L9 and L12, 40 nM for L11, and ˜3 μM for L15 (FIG. 2C). As expected both control domain antibodies failed to demonstrate any kinase blocking activity in this assay. In addition, 1 μM of all 6 purified domain antibodies failed to bind or block the kinase activity of EGFR, KDR, FGFR1, PDGFRβ, Fyn, Fms, c-Met, insulin-receptor, Src, and Btx or Itk (two Tec family members) (data not shown). These findings indicate that the neutralizing anti-Etk domain antibodies possess high specificity and good kinase blocking activity, and therefore, may have a potential to serve as intracellular antibodies to block endogenous Etk enzymatic activity and interfere with Etk-related signaling cascades.

Construction and expression of intrabodies. DNA coding for domain antibodies were amplified and subcloned into the Hind III and EcoR1 sites of pcDNA3.1 vector (Invitrogen). NSR cells were transfected with 1 of the 6 constructs coding for different intrabodies using the Lipofectamine method (Duzgunes, N. et al., Methods Enzymol. 221:303-6, 1993). Due to low transfection yield common to NIH3T3 cells (10%), transfected cells were selected.

The cells were transferred into a medium containing 10% FCS and supplemented with 1 μg/ml Geneticin G418 at 24 h post transfection. Survivor colonies were pooled gradually up scaled during a period of 4 weeks, and used for various assays. In total, 6 domain antibodies were expressed as intrabodies (clones designated K5, K7, K9, K11, K12 and K15) in NSR cells.

Western immunoblotting and immunoprecipitation. Cells from a 150 mm dish were harvested in 1 ml lysis buffer containing 25 mM Tris-HCl pH 7.4, 2 mM sodium orthovanadate, 0.5 mM EDTA, 10 mM NaF, 10 mM sodium pyrophosphate, 25 mM NaCl and 1% Tx-100 supplemented with 1:1000 dilution of protease inhibitor cocktail. Cells were frozen and thawed twice, centrifuged and supernatant was collected. Samples of 50 μg protein extract were resolved by SDS-PAGE under reducing conditions, and transferred onto nitrocellulose membrane for western immunoblotting with the indicated antibodies. For immunoprecipitation, 0.5 mg of protein extracts were incubated with either protein L beads or anti-Etk antibodies coupled to protein A bead overnight at 4° C. The immunocomplex was washed 4 times with PBST and twice with 50 mM Hepes, pH 7.5, electrophored on an SDS-polyacrylamide gel under reducing conditions, and transferred onto nitrocellulose membrane for western immunoblotting with the indicated antibodies. In addition, the immunocomplex was also used as the source for enzyme (Etk kinase) in the in vitro kinase activity and autophosphorylation assays described below.

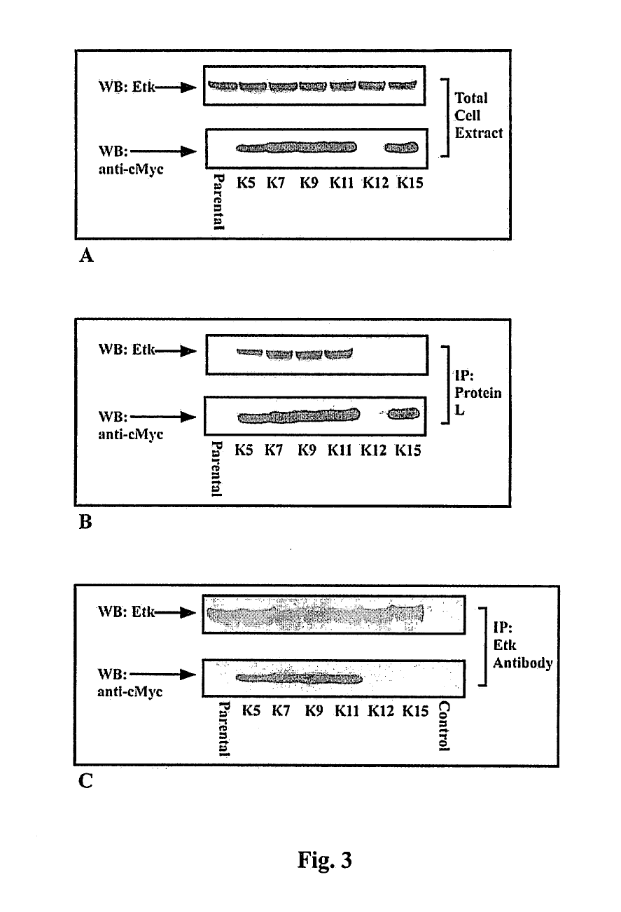

As shown in FIG. 3A, 5 out of 6 clones, K5, K7, K9, K11 and K15, highly expressed intrabodies, as demonstrated by a single protein band corresponding to the expected molecular size of 12 kDa when detected by an anti-c-Myc antibody. In contrast, cells transfected with K12 failed to produce any detectable intrabody (FIG. 3A). Only 4 out of the 5 expressed intrabodies (K5, K7, K9 and K11) were capable of interacting with endogenous Etk as demonstrated by immunoprecipitation of the cell lysate with immobilized protein L, which is proficient in binding the kappa light chain domain antibodies, followed by blotting with an anti-Etk antibody (FIG. 3B). This was further confirmed when the cell lysate was immunoprecipitated with an anti-Etk antibody followed by blotting with an anti-c-Myc antibodies (FIG. 3C). While K15 intrabody expressed well in NSR cells, it failed to interact with endogenous Etk (FIG. 3). These results indicate that the intracellularly expressed K5, K7, K9 and K11 intrabodies fold appropriately, thus allowing them to interact with their target in the cytoplasm compartment.

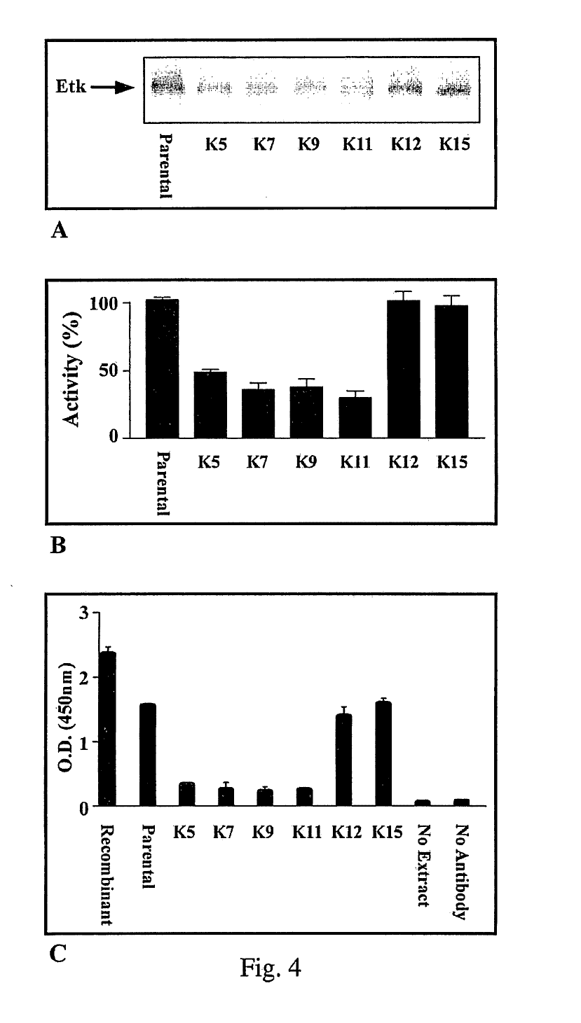

In vitro autophosphorylation assay. Etk from both the intrabody-transfected cells and the parental NSR cells were immunoprecipitated using an anti-Etk antibody, and subjected to an in vitro autophosphorylation assay using [γ-³³P] ATP. The immunocomplex, in a final volume of 30 μl was mixed with 10 μl of 4× phosphorylation buffer containing 200 mM Hepes pH 7.5, 20 mM MgCl₂, 20 mM MnCl₂, 2 mM DTT, and 1 μM [γ-³³P]ATP. The reaction was allowed to proceed for 15 min at RT, and was terminated by adding 10 μl of 4×SDS-PAGE sample buffer. Samples were resolved by SDS-PAGE under reducing conditions and were subjected to autoradiography. As shown in FIG. 4A, Etk derived from clones K5, K7, K9 and K11 demonstrated a significantly lower rate of tyrosine autophosphorylation, retaining approximately 49%, 37%, 38% and 30% of the wild-type enzyme (from NSR cells) activity, respectively. Consistent with expression and binding data, Etk from cells expressing clones K12 (no expression) and K15 (no Etk interaction) showed kinase activity comparable to that of Etk derived from NSR cells (FIG. 4A).

In vitro kinase assays. Similar results were observed when endogenous Etk was used as the source of kinase for a substrate polyEY in a quantative in vitro kinase assay. Etk/intrabody immunocomplexes derived from NSR cells expressing the intrabody (obtained by immunoprecipitation, see above) were incubated with the polyEY, and the amount of substrate phosphorylation was determined. The enzymatic activity of Etk derived from clones K5, K7, K9 and K11 was reduced to approximately 25% of that observed with Etk derived from NSR cells. In contrast, Etk derived from clones K12 and K15 showed similar levels of enzymatic activity to that derived from NSR cells (FIG. 4B).

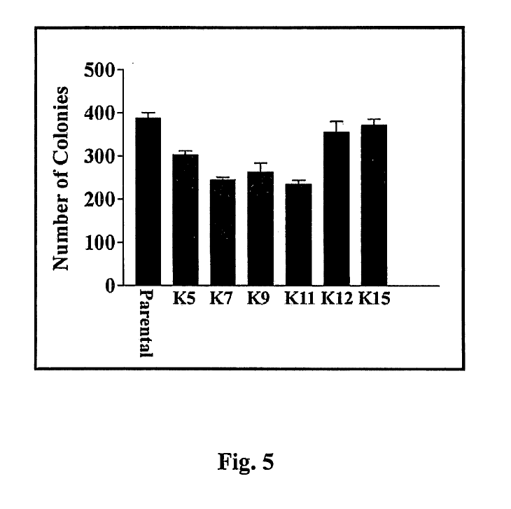

Effect of anti-Etk intrabodies on cell transformation. When seeded and grown as an adherent culture, the overall growth rate of the domain antibody-transfected clones was comparable to that of the parental NSR cells. the effect of the anti-Etk intrabodies on Src-induced cellular transformation was studied by comparing colony formation in soft agar of parental NSR cells and intrabody-transfected cells. Soft-agar assays were performed as previously described, (Paz, K. et al., Oncogene 23:8455-63, 2004). Briefly, lower layer (in 35 mm diameter dishes) of 0.7% low melting agar solution in DMEM supplemented with 0.10% FCS, was covered by a second layer of 0.35% agar solution in DMEM, in which 10⁵ cells were resuspended. Following solidification, growth medium (DMEM supplemented with 10% FCS) was added to the dishes. The dishes were incubated at 37° C. for 14 days, with growth medium changes every three days. Colonies were stained with 1 mg/ml MTT in PBS and scored using AlphaEaseFC program (FIG. 5).

Although colonies of 50 or more cells were observed with both parental NSR cells and cells expressing the intrabodies, the NSR cells showed higher colony formation efficiency with 387±25 colonies per plate. Cells transfected with K5, K7, K9 or K11 showed colony formation rate of 302±19 (p=0.001), 245±6 (p=0.0), 262±43 (p=0.069) and 234±18 (p=0.002), respectively. These numbers are significantly lower than that observed with NSR cells, and represent a 22%, 37%, 31% and 40% reduction in colony formation for clones K5, K7, K9 and K11 respectively. As expected, both clone K12 and clone K15 showed a similar growth and colony formation rate to NSR cells, with 354±24 and 371±26 colonies per plate, respectively (FIG. 5).

### REFERENCES

1. Alvarez, R. D. et al., 2000, A cancer gene therapy approach utilizing an anti-erbB-2 single-chain antibody-encoding adenovirus (AD21): a phase I trial. Clin. Cancer Res. 6:3081-7.
2. Arafat, W. et al, 2000, Antineoplastic effect of anti-erbB-2 intrabody is not correlated with scFv affinity for its target. Cancer Gene Ther. 7:1250-6.
3. Richardson, J. H. et al., 1995, Phenotypic knockout of the high-affinity human interleukin 2 receptor by intracellular single-chain antibodies against the alpha subunit of the receptor. Proc. Natl. Acad. Sci. U.S.A. 92:3137-41.
4. Strube, R. W. et al., 2002, Characterization of anti-cyclin E single-chain Fv antibodies and intrabodies in breast cancer cells: enhanced intracellular stability of novel sFv-F(c) intrabodies. J. Immunol. Methods 263:149-67.
5. Lecerf, J. M. et al., 2001, Human single-chain Fv intrabodies counteract in situ huntingtin aggregation in cellular models of Huntington's disease. Proc. Natl. Acad. Sci. U.S.A. 98:4764-9.
6. Marasco, W. A., 2001, Intrabodies as antiviral agents. Curr. Top. Microbiol. Immunol. 0.260:247-70.
7. Stocks M R., 2004, Intrabodies: production and promise. Drug. Discov. Today 9:960-6.
8. Smith, C. I. et al, 2001, The Tec family of cytoplasmic tyrosine kinases: mammalian Btk, Bmx, Itk, Tec, Txk and homologs in other species. Bioessays 23:436-46.
9. Tomlinson, M. G. et al, 2004, Expression and function of Tec, Itk, and Btk in lymphocytes: evidence for a unique role for Tec. Mol Cell Biol 24:2455-66.
10. Qiu, Y. et al., 2000, Signaling network of the Btk family kinases. Oncogene 19:5651-61.
11. Kerner, J. D. et al., Impaired expansion of mouse B cell progenitors lacking Btk. Immunity 3:301-12.
12. Khan, W. N. et al., 1995, The role of Bruton's tyrosine kinase in B-cell development and function in mice and man. Ann. N.Y. Acad. Sci. 764:27-38.
13. Rawlings, D. J. et al., 1993, Mutation of unique region of Bruton's tyrosine kinase in immunodeficient MD mice. Science 261:358-61.
14. Tsukada, S. et al., 1994, X-linked agammaglobulinemia and Bruton's tyrosine kinase. Adv. Exp. Med. Biol. 365:233-8.
15. Tamagnone, L. et al., 1994, BMX, a novel nonreceptor tyrosine kinase gene of the BTK/ITK/TEC/TXK family located in chromosome Xp22.2. Oncogene 9:3683-8.
16. Robinson, D. et al., 1996, A tyrosine kinase profile of prostate carcinoma. Proc. Natl. Acad. Sci. USA 93:5958-62.
17. Qiu Y. et al., 1998, Etk/Bmx, a tyrosine kinase with a pleckstrin-homology domain, is an effector of phosphatidylinositol 3′-kinase and is involved in interleukin 6-induced neuroendocrine differentiation of prostate cancer cells. Proc. Natl. Acad. Sci. U S A 95:3644-9.
18. Tsai, Y. T. et al., 2000, Etk, a Btk family tyrosine kinase, mediates cellular transformation by linking Src to STAT3 activation. Mol. Cell. Biol. 20:2043-54.
19. Chen, K. Y. et al., 2004, The role of tyrosine kinase Etk/Bmx in EGF-induced apoptosis of MDA-MB-468 breast cancer cells. Oncogene 23:1854-62.
20. Chau, C. H. et al., 2002, Coordinating Etk/Bmx activation and VEGF upregulation to promote cell survival and proliferation. Oncogene 21:8817-29.
21. Chen, R. et al., 2001, Regulation of the PH-domain-containing tyrosine kinase Etk by focal adhesion kinase through the FERM domain. Nat. Cell. Biol. 3:439-44.
22. Jiang, T. et al., 2004, Bi-directional regulation between tyrosine kinase Etk/BMX and tumor suppressor p53 in response to DNA damage. J. Biol. Chem. 279:50181-9.
23. Bagheri-Yarmand, R. et al., 2001, Etk/Bmx tyrosine kinase activates Pak1 and regulates tumorigenicity of breast cancer cells. J. Biol. Chem. 276:29403-9.
24. Paavonen, K. et al., 2004, Bmx tyrosine kinase transgene induces skin hyperplasia, inflammatory angiogenesis, and accelerated wound healing. Mol. Biol. Cell. 15:4226-33.
25. Lee, L. F. et al., 2001, Neuropeptide-induced androgen independence in prostate cancer cells: roles of nonreceptor tyrosine kinases Etk/Bmx, Src, and focal adhesion kinase. Mol. Cell. Biol. 21:8385-97.
26. Xue, L. Y. et al., 1999, Etk/Bmx, a PH-domain containing tyrosine kinase, protects prostate cancer cells from apoptosis induced by photodynamic therapy or thapsigargin. Oncogene 18:3391-8.
27. Frame M C., 2004, Newest findings on the oldest oncogene; how activated src does it. J. Cell. Sci. 117:989-98.
28. Ishizawar, R. et al., 2004, c-Src and cooperating partners in human cancer. Cancer Cell 6:209-14.
29. He, H. et al., 2004, The Tyr-kinase inhibitor AG879, that blocks the ETK-PAK1 interaction, suppresses the RAS-induced PAK1 activation and malignant transformation. Cancer Biol. Ther. 3:96-101.
30. Ellis, L. et al., 1988, Efficient expression in insect cells of a soluble, active human insulin receptor protein-tyrosine kinase domain by use of a baculovirus vector. J. Virol. 62:1634-9.
31. McNew, J. A. et al., 2000, Compartmental specificity of cellular membrane fusion encoded in SNARE proteins. Nature 407:153-9.
32. Lu, D. et al., 2002, Selection of high affinity human neutralizing antibodies to VEGFR2 from a large antibody phage display library for antiangiogenesis therapy. Int. J. Cancer 97:393-9.
33. Duzgunes, N. et al., 1993, Intracellular delivery of nucleic acids and transcription factors by cationic liposomes. Methods Enzymol. 221:303-6.
34. Paz, K. et al., 2004, Transformation fingerprint: induced STAT3-C, v-Src and Ha-Ras cause small initial changes but similar established profiles in mRNA. Oncogene 23:8455-63.
35. Tanaka, T. et al., 2003, Single domain intracellular antibodies: a minimal fragment for direct in vivo selection of antigen-specific intrabodies. J. Mol. Biol. 331:1109-20.
36. Cohen, P. A. et al., 2002, Intrabodies. Targeting scFv expression to eukaryotic intracellular compartments. Methods Mol. Biol. 178:367-78.
37. Wheeler, Y. Y. et al., 2003, Intrabody and intrakine strategies for molecular therapy. Mol. Ther. 8:355-66.
38. Biocca, S. et al., 1995, Redox state of single chain Fv fragments targeted to the endoplasmic reticulum, cytosol and mitochondria. Biotechnology (NY) 13:1110-5.
39. Proba, K. et al., 1998, Antibody scFv fragments without disulfide bonds made by molecular evolution. J. Mol. Biol. 275:245-53.
40. Donini, M. et al., 2003, Engineering stable cytoplasitic intrabodies with designed specificity. J. Mol. Biol. 330:323-32.
41. Ewert, S. et al, 2003, Biophysical properties of human antibody variable domains. J. Mol. Biol. 325:531-53.
42. Wirtz, P. et al., 1999, Intrabody construction and expression III: engineering hyperstable V(H) domains. Protein Sci. 8:2245-50.
43. Worn, A. et. al., 1998, Mutual stabilization of VL and VH in single-chain antibody fragments, investigated with mutants engineered for stability. Biochemistry 37:13120-7.
44. Tanaka, T. et al., 2003, Intrabodies based on intracellular capture frameworks that bind the RAS protein with high affinity and impair oncogenic transformation. EMBO J. 22:1025-35.
45. Tse, E. et al., 2002, Intracellular antibody capture technology: application to selection of intracellular antibodies recognising the BCR-ABL oncogenic protein. J. Mol. Biol. 317:85-94.
46. Visintin, M. et al., 2002, The intracellular antibody capture technology (IACT): towards a consensus sequence for intracellular antibodies. J. Mol. Biol. 317:73-83.
47. Marasco, W. A. et al., 1998, Intracellular antibodies against HIV-1 envelope protein for AIDS gene therapy. Hum. Gene Ther. 9:1627-42.
48. Marasco, W. A. et al., 1993, Design, intracellular expression, and activity of a human anti-human immunodeficiency virus type 1 gp120 single-chain antibody. Proc. Natl. Acad. Sci. USA 90:7889-93.
49. Dougall, W. C. et al., 1994, Biological studies and potential therapeutic applications of monoclonal antibodies and small molecules reactive with the neu/c-erbB-2 protein. Cell. Biophys. 24-25:209-18.
50. Hyland, S. et al., 2003, Generation and functional characterization of intracellular antibodies interacting with the kinase domain of human EGF receptor. Oncogene 22:1557-67.
51. Bai, J. et al., 2003, Inhibition of Tat-mediated transactivation and HIV-1 replication by human anti-hCyclinT1 intrabodies. J. Biol. Chem. 278:1433-42.
52. Xu, D. et al., 2002, [Effects of anti-ABL tyrosine kinase intrabody on the growth of K562 cells in nude mice]. Zhongguo Yi Xue Ke Xue Yuan Xue Bao 24:11-4.
53. Zhu, Q. et al., 1999, Extended half-life and elevated steady-state level of a single-chain Fv intrabody are critical for specific intracellular retargeting of its antigen, caspase-7. J. Immunol. Methods 231:207-22.
54. Wheeler, Y. Y. et al., 2003, Intrabody-based strategies for inhibition of vascular endothelial growth factor receptor-2: effects on apoptosis, cell growth, and angiogenesis. FASEB J. 17:1733-5.
55. Thou, P. et al., 2000, Harnessing the ubiquitination machinery to target the degradation of specific cellular proteins. Mol. Cell. 6:751-6.

1. A single domain intrabody that binds to an intracellular protein or to an intracellular domain of an intracellular protein.

2. The single domain intrabody of claim 1 that binds to an intracellular enzyme.

3. The single domain intrabody of claim 1 that binds to a kinase domain of Etk.

4. The single domain intrabody of claim 3 that is a human kappa chain variable domain antibody comprising one, two, or three complementarity determining regions selected from the group consisting of SEQ ID NO:4, SEQ ID NO:6, and SEQ ID NO:8 at CDR1, CDR2, and CDR3 respectively.

5. The single domain intrabody of claim 3 that is a human kappa chain variable domain antibody comprising one, two, or three complementarity determining regions selected from the group consisting of SEQ ID NO:12, SEQ ID NO:14, and SEQ ID NO:16 at CDR1, CDR2, and CDR3 respectively.

6. The single domain intrabody of claim 3 that is a human kappa chain variable domain antibody comprising one, two, or three complementarity determining regions selected from the group consisting of SEQ ID NO:20, SEQ ID NO:22, and SEQ ID NO:24 at CDR1, CDR2, and CDR3 respectively.

7. The single domain intrabody of claim 3 that is a human kappa chain variable domain antibody comprising one, two, or three complementarity determining regions selected from the group consisting of SEQ ID NO:28, SEQ ID NO:30, and SEQ ID NO:32 at CDR1, CDR2, and CDR3 respectively.

8. The single domain intrabody of claim 3 that is a human kappa chain variable domain antibody comprising one, two, or three complementarity determining regions selected from the group consisting of SEQ ID NO:36, SEQ ID NO:38, and SEQ ID NO:40 at CDR1, CDR2, and CDR3 respectively.

9. The single domain intrabody of claim 3 that is a human kappa chain variable domain antibody comprising one, two, or three complementarity determining regions selected from the group consisting of SEQ ID NO:44, SEQ ID NO:46, and SEQ ID NO:48 at CDR1, CDR2, and CDR3 respectively.

10. The single domain intrabody of claim 3 that is a human kappa chain variable domain antibody comprising a complementarity determining region selected from the group consisting SEQ ID NO:2, SEQ ID NO:10, SEQ ID NO:18, SEQ ID NO:26, SEQ ID NO:34, and SEQ ID NO:42.

11. A method of inhibiting intracellular enzyme activity in a cell, comprising introducing the intracellular intrabody of claim 1 into the cytoplasm of the cell, wherein the intrabody binds to an intracellular enzyme in the cell, thereby inhibiting intracellular activity in the cell.

12. The method of claim 11 wherein the intracellular enzyme is Etk.

13. The method of claim 11 wherein the step of introducing comprises causing a nucleic acid molecule expressing the intracellular intrabody of claim 1 to enter the cell.

14. A method of treating or inhibiting a tumor in a patient comprising administering, to a patient in need thereof, the intracellular intrabody of claim 1 or a nucleic acid molecule expressing the intrabody.

15. The method of claim 14 wherein the step of administering is via a route selected from the group consisting of oral, intravenous, intraperitoneal, subcutaneous, and intramuscular administration.

16. The method of claim 14 that further comprises co-administering at least one additional anticancer therapeutic modality.

17. The method of claim 16 wherein the additional anticancer therapeutic modality is an anti-neoplastic agent or radiation.

18. The method of claim 17 wherein the anti-neoplastic agent is an alkylating agent or an anti-metabolite.

19. The method of claim 18 wherein the alkylating agent is selected from the group consisting of cisplatin, cyclophosphamide, melphalan, and dacarbazine.

20. The method of claim 18 wherein the anti-metabolite is selected from the group consisting of doxorubicin, daunorubicin, paclitaxel and gemcitabine.
